(19) United States
(12) Patent Application Publication
(10) Pub. No.: US 2010/0131049 A1
(43) Pub. Date: May 27, 2010

(54) One-Way valve Prosthesis for Percutaneous Placement Within the Venous System

(75) Inventors: D.H. Perkins, Santa Rosa, CA (US); Dustin Thompson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/276,866

(22) Filed: Nov. 24, 2008

(51) Int. Cl. A61F 2/06; A61F 2/04

(52) U.S. Cl. 623/1.24; 623/23.7

(57) ABSTRACT

A one-way valve prosthesis for percutaneous placement within a vein, the valve including a valve body having an inlet and an outlet with a lumen that extends there between. The valve body is operable to alternate between a closed configuration wherein the valve body has a double cone shape and an open configuration wherein the valve body has a double frustoconical shape. A valve seat if formed within the lumen of the valve body at a midsection thereof. The valve seat is constricted to prevent flow there through when the valve body is in the closed configuration and the valve seat is open to allow flow there through when the valve body is in the open configuration. The valve seat opens in response to an actuation pressure and closes in the absence of the actuation pressure.

### FIELD OF THE INVENTION

The invention relates to valve prostheses for percutaneous placement within a vein.

### BACKGROUND OF THE INVENTION

Venous valves are found within native venous vessels and are used to assist in returning blood back to the heart in an antegrade direction from all parts of the body. The venous system of the leg for example includes the deep venous system and the superficial venous system, both of which are provided with venous valves which are intended to direct blood toward the heart and prevent backflow or retrograde flow which can lead to blood pooling or stasis in the leg. Incompetent valves can also lead to reflux of blood from the deep venous system to the superficial venous system and the formation of varicose veins. Superficial veins which include the greater and lesser saphenous veins have perforating branches in the femoral and popliteal regions of the leg that direct blood flow toward the deep venous system and generally have a venous valve located near the junction with the deep system. Deep veins of the leg include the anterior and posterior tibial veins, popliteal veins, and femoral veins. Deep veins are surrounded in part by musculature tissues that assist in generating flow due to muscle contraction during normal walking or exercising. Veins in the lower leg of a healthy person may range from 0 mm Hg to over 200 mm Hg, depending on factors such as the activity of the body (i.e., stationary or exercising), the position of the body (i.e., supine or standing), and the location of the vein (i.e., ankle or thigh). For example, venous pressure may be approximately 80-90 mm Hg while standing and may be reduced to 60-70 mm Hg during exercise. Despite exposure to such pressures, the valves of the leg are very flexible and can close with a pressure drop of less than one mm Hg.

FIGS. 1A-1B are schematic representations of blood flow through a healthy native valve 104 within a vein 100. Valves within the venous system are configured in a variety of shapes that depend on anatomical location, vessel size, and function. For example, the shape of the venous valve may include leaflets or leaflets with sinuses. The natural venous valve leaflet configuration referenced herein is for clarity of function and is not limiting in the application of the referenced embodiments. Venous valve 104 controls blood flow through lumen 102 of vein 100 via leaflets 106, 108. More particularly, venous valve 104 opens to allow antegrade flow 112 through leaflets 106, 108 as shown in FIG. 1A. Venous valve 104 closes to prevent backflow or retrograde flow 114 through leaflets 106, 108 as shown in FIG. 1B.

Veins typically in the leg can become distended from prolonged exposure to excessive pressure and due to weaknesses found in the vessel wall causing the natural venous valves to become incompetent leading to retrograde blood flow in the veins. Such veins no longer function to help pump or direct the blood back to the heart during normal walking or use of the leg muscles. As a result, blood tends to pool in the lower leg and can lead to leg swelling and the formation of deep venous thrombosis and phlebitis. The formation of thrombus in the veins can further impair venous valvular function by causing valvular adherence to the venous wall with possible irreversible loss of venous function. Continued exposure of the venous system to blood pooling and swelling of the surrounding tissue can lead to post phlebitic syndrome with a propensity for open sores, infection, and may lead to limb amputation.

Chronic Venous Insufficiency (CVI) occurs in patients that have deep and superficial venous valves of their lower extremities (distal to their pelvis) that have failed or become incompetent due to congenital valvular abnormalities and/or pathophysiologic disease of the vasculature. As a result, such patients suffer from varicose veins, swelling and pain of the lower extremities, edema, hyper pigmentation, lipodermatosclerosis, and deep vein thrombosis (DVT). Such patients are at increased risk for development of soft tissue necrosis, ulcerations, pulmonary embolism, stroke, heart attack, and amputations.

FIG. 2 is a schematic representation of blood flow through an incompetent venous valve. Backflow or antegrade flow 114 leaks through venous valve 104 creating blood build-up that eventually may destroy the venous valve and cause a venous wall bulge 110. More specifically, the vessel wall of vein 100 expands into a pouch or bulge, such that the vessel has a knotted appearance when the pouch is filled with blood. The distended vessel wall area may occur on the outflow side of the valve above leaflets 106, 108 as shown in FIG. 2, and/or on the inflow side of the valve below leaflets 106, 108. After a vein segment becomes incompetent, the vessel wall dilates and fluid velocity there through decreases, which may lead to flow stasis and thrombus formation in the proximity of the venous valve. Repair and replacement of venous valves presents a formidable challenge due to the low blood flow rate found in native veins, the very thin wall structure of the venous wall and the venous valve, and the ease and frequency of which venous blood flow can be impeded or totally blocked for a period of time. Surgical reconstruction techniques used to address venous valve incompetence include venous valve bypass using a segment of vein with a competent valve, venous transposition to bypass venous blood flow through a neighboring competent valve, and valvuloplasty to repair the valve cusps. These surgical approaches may involve placement of synthetic, allograft and/or xenograft prostheses inside of or around the vein. However, such prostheses have not been devoid of problems, such as thrombus formation and valve failure due to the implanted prostheses causing non-physiologic flow conditions and/or excessive dilation of the vessels with a subsequent decrease in blood flow rates. In addition, many venous valve prostheses include leaflets and/or hinged flaps and are similar to valves placed into the heart, which are complex and designed for high blood pressures associated with the heart instead of lower venous blood pressures associated with veins in the lower extremities.

Percutaneous methods for treatment of venous insufficiency are being studied some of which include placement of synthetic, allograft and/or xenograft prosthesis that suffer from similar problems as the surgically implanted ones discussed above.

In light of these limitations, there is a need for an improved device to restore normal venous circulation to patients suffering from venous valve insufficiency. The present disclosure is directed to a simple, one-way valve prosthesis that may be percutaneously placed within a vein to replace an existing insufficient venous valve. After placement, the valve prosthesis re-establishes proper flow through the vein segment and protects any damaged area(s) of the native valve for healing.

### BRIEF SUMMARY OF THE INVENTION

Embodiments hereof are directed to a one-way venous valve prosthesis for percutaneous placement within a vein, the valve including a valve body having an inlet and an outlet with a lumen that extends there between. The valve body is operable to alternate between a closed configuration wherein the valve body has a double cone shape and an open configuration wherein the valve body has a double frustoconical shape. When the valve body is in the double cone shape, conical apexes are located at a midsection of the valve body and define a valve seat within the lumen of the valve body. The valve seat is constricted to prevent flow there through when the valve body is in the double cone shape of the closed configuration and the valve seat is open to allow flow there through when the valve body assumes the double frustoconcial shape in the open configuration. The valve seat expands to the open configuration in response to an actuation pressure and returns to the closed configuration in the absence of the actuation pressure.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIGS. 1A-1B are schematic representations of blood flow through a healthy valve within a vein.

FIG. 2 is a schematic representation of blood flow through an incompetent valve within a vein.

FIG. 3 is a perspective view of a double cone valve prosthesis according to an embodiment hereof, wherein the valve prosthesis is in a closed configuration.

FIG. 4 is a side view of the double cone valve prosthesis shown in FIG. 3, wherein the valve prosthesis is in an open configuration.

FIG. 5 is an end view of the double cone valve prosthesis shown in FIG. 4.

FIG. 6 is a schematic sectional view of an incompetent valve within a vein.

FIG. 7 is a schematic view of the double cone valve prosthesis shown in FIG. 3 placed within the incompetent valve of FIG. 6, wherein the double cone prosthesis is in the closed configuration to prevent blood flow there through.

FIG. 8 is a schematic view of the double cone valve prosthesis shown in FIG. 3 placed within the incompetent valve of FIG. 6, wherein the double cone prosthesis is in the open configuration to allow blood flow there through.

FIG. 9 is a side view of a double cone valve prosthesis according to an embodiment hereof.

FIG. 10 is a side view of a double cone valve prosthesis according to another embodiment hereof.

FIG. 11 is a side view of a double cone valve prosthesis according to yet another embodiment hereof.

FIGS. 12-13 are a side view and a perspective view, respectively, of a double cone valve prosthesis according to yet another embodiment hereof.

FIG. 14 is a perspective view of a double cone valve prosthesis having self-expanding anchors according to yet another embodiment hereof.

FIG. 15 is an example of a delivery system for delivering a double cone valve prosthesis.

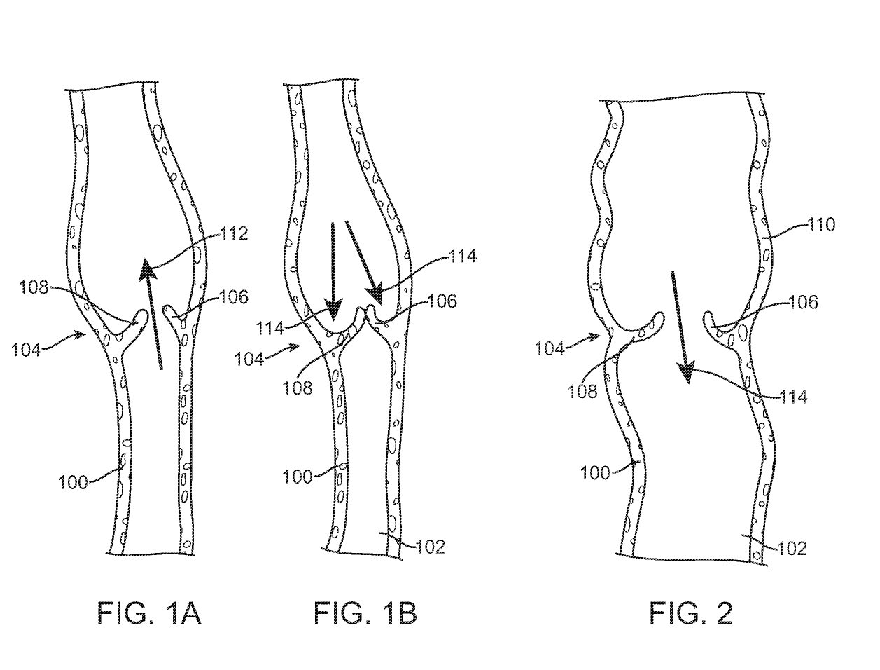

### DETAILED DESCRIPTION OF THE INVENTION

Specific embodiments hereof are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the veins, the invention may also be used in any other body passageways where it is deemed useful. Furthermore, there is no intention to be bound by any expressed or implied theory presented in the preceding technical field, background, brief summary or the following detailed description.

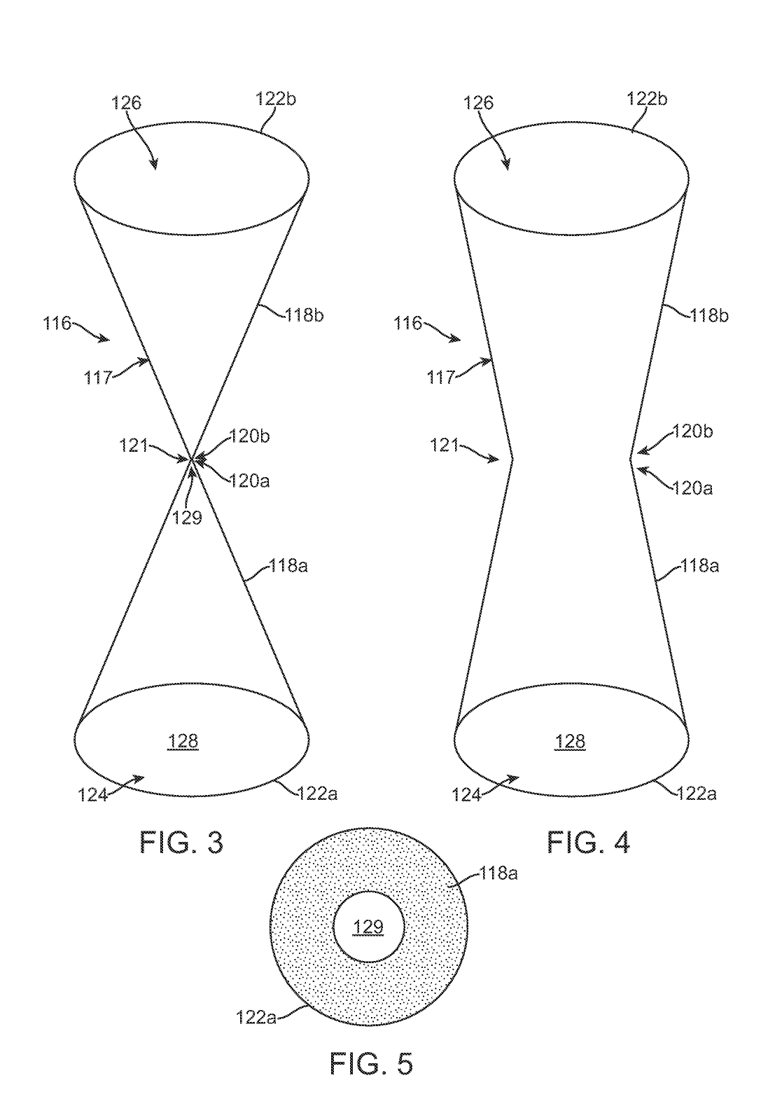

Referring to FIGS. 3-5, a venous valve 116 according to an embodiment hereof is shown. Valve 116 has a continuous body portion 117 that defines a lumen 128 extending between an inlet 124 and an outlet 126. Valve 116 is operable to alternate between a closed configuration, shown in FIG. 3, in which a midsection 121 of valve body 117 is constricted to prevent flow there through and an open configuration, shown in FIG. 4, in which midsection 121 is open or expanded enough to allow flow there through. FIG. 3 illustrates venous valve 116 in a relaxed state, which is the valve closed configuration, and FIGS. 4-5 illustrate venous valve 116 in a working or flow state, which is the valve open configuration.

More particularly, in the closed configuration illustrated in FIG. 3, body portion 117 of venous valve 116 has a continuous double cone shape having a first conical section 118a and a second conical section 118b that are oriented apex to apex. In an embodiment, a length of first conical section 118a is equal to a length of second conical section 118b. However, conical sections 118a, 118b may be of unequal lengths. From a downstream end, first conical section 118a has a cone shape that extends from a first circular base 122a, which defines inlet 124 of valve 116, to a first apex 120a, and second conical section 118b has a cone shape that extends from a second apex 120b to a second circular base 122b, which defines outlet 126 of valve 116. It should be understood that first apex 120a and second apex 120b are so defined at midsection 121 of valve body portion 117 only in the closed, relaxed configuration when venous valve 116 has the double cone shape. At a corresponding location of apexes 120a, 120b within lumen 128 of valve body portion 117, a valve seat 129 is defined. When valve 116 is in the closed configuration, valve seat 129 is constricted or closed in such a manner as to prevent flow there through.

Referring now to FIGS. 4-5, when venous valve 116 is in the open configuration, valve body portion 117 has a continuous double frustoconical shape. From a downstream end, first conical section 118a assumes a frustoconical shape that extends from first circular base 122a to radially expanded apex 120a, and second conical section 118b assumes a frustoconical shape that extends from radially expanded apex 120b to second circular base 122b. As in the closed configuration, the radially expanded apexes 120a, 120b of conical sections 118a, 118b are defined/located at midsection 121 of valve body portion 117, such that venous valve 116 has the double frustoconical shape in the open configuration. In the open configuration, valve seat 129, which as previously described is defined within lumen 128 of valve body portion 117 at midsection 121, is radially expanded to allow flow there through, as best shown in the end view of FIG. 5.

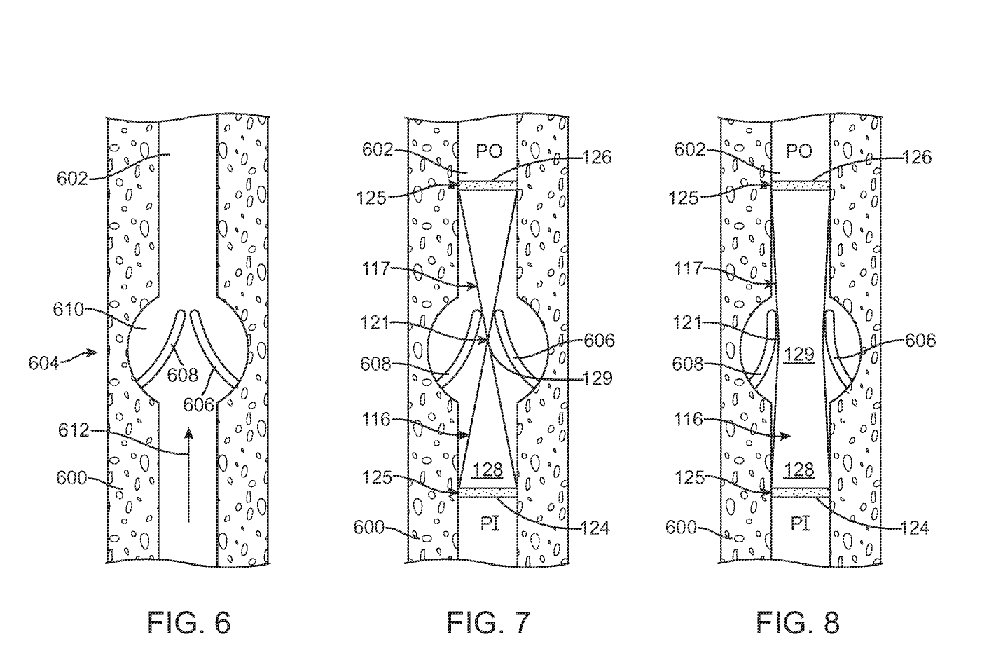

Referring now to FIGS. 6-8, the operation of valve 116 transitioning between the closed configuration and the open configuration for regulating flow there through is described. FIG. 6 is an illustration of an incompetent valve 604 of a vein 600. Valve 604 includes two leaflets 606, 608, for controlling blood flow through lumen 602 of vein 600 in an antegrade direction indicated by directional arrow 612. However, valve leaflets 606, 608 do not completely close and thus allow some venous blood to flow in a retrograde direction. The backflow causes a distended area or bulge 610, which is a localized area of blood pooling that creates a bulging of the venous wall. As the bulging progresses, vein 600 becomes further enlarged and valve leaflets 606, 608 move farther apart, allowing even more blood to backflow. Thus, once valve 604 becomes incompetent, the venous insufficiency/incompetency progressively worsens.

FIG. 7 is a schematic view of venous valve 116 placed within incompetent valve 604 of vein 600. Valve 116 is delivered to and deployed within vein 600 in a percutaneous manner, as will be described in more detail below, and is positioned to span across valve leaflets 606, 608 of incompetent valve 604. Although not shown in FIGS. 6-8, valve body portion 117 may include one or more radiopaque or echogenic markers attached thereto to assist in positioning valve 116 across incompetent valve 614. Thus, prosthetic valve 116 may be implanted without requiring removal of native valve 604 from vein 600. In addition, since valve body 117 of venous valve 116 spans across insufficient valve 604, valve 116 will arrest the progressive damage to vein 600 caused by the marginal function of native valve 604. Blood flow will then be directed through lumen 128 of valve 116 and thus bypass distended or bulged area 610. The damaged venous wall will thus be protected and allowed to scar and/or heal.

FIG. 7 illustrates valve 116 in the closed configuration described above with respect to FIG. 3, in which valve 116 has a double cone shape such that midsection 121 of valve body 117 is constricted or closed to prevent flow there through. As shown in FIG. 7, valve 116 is secured to the wall of vein 600 by one or more anchors 125. In one embodiment, an anchor 125 is attached to each end of valve 116 such that inlet 124 of valve 116 is secured to the vessel wall and outlet 126 of valve 116 is secured to the vessel wall. Anchors 125 are annular, self-expanding structures that are attached to valve 116 in order to prevent migration thereof. For example, anchors 125 may be self-expanding spring members that are deployed upon release from a restraining mechanism such as a retractable sheath to bias valve 116 into conforming fixed engagement with an interior surface of vein 600. Anchors 125 may be constructed of a superelastic material such as nickel-titanium (nitinol) and have any suitable configuration. For example, anchors 125 may be annular bands as shown in FIGS. 7-8 biased in a radially outward direction. Alternatively, anchors 125 may be sinusoidal patterned wire rings or scaffolds 1425 biased in a radially outward direction as shown in FIG. 14. Examples of suitable annular support members that may be used as anchors 125 are described, for example, in U.S. Pat. No. 5,713,917 to Leonhardt et al. and U.S. Pat. No. 5,824,041 to Lenker et al., which are incorporated by reference herein in their entirety. When used with valve 116, anchors 125 have sufficient radial spring force and flexibility to conformingly engage the prosthesis with the body lumen inner wall, to avoid excessive leakage, and prevent pressurization of the native valve, i.e., to provide a leak-resistant seal.

Once implanted in vein 600, venous valve 116 operates as a one-way valve that allows fluid to flow in only an antegrade direction in order to control blood flow through lumen 602 of vein 600. Once the pressure on the inflow area of valve 116 reaches and/or exceeds an actuation pressure PA, valve 116 expands to the open configuration. The actuation pressure PA is related to the pressure differential that occurs during normal blood circulation between the pumped blood on the valve inflow area and the gravity fed blood on the valve outflow area to allow valve 116 to operate in a manner similar to a natural venous valve. More particularly, when the pumped blood causes the inflow pressure to reach a value equal to or greater than the combination of the gravity fed blood pressure and the valve's resistance to opening, i.e., the actuation pressure PA, valve 116 opens in response thereto. The valve's resistance to opening may depend on several factors, including the stiffness of the valve material, the thickness of the valve material, and/or the geometry of the valve inflow and outflow areas. By manipulating these factors, valve 116 may be designed to open under inflow pressure conditions that depend on the particular implantation site of the prosthetic valve within the vasculature. As will be described in more detail below, valve 116 is constructed such that midsection 121 of valve body 117 expands to the open configuration in which valve seat 129 of lumen 128 is sufficiently open to accommodate flow there through in response to actuation pressure PA. In the absence of actuation pressure PA, such as during normal pauses of blood circulation through the body, valve seat 129 resumes the closed configuration. The relatively simple construction of venous valve 116 does not include leaflets or hinged flaps that may thicken, tear or fail, avoids tissue ingrowth of such leaflets, and also avoids pooling of blood within such leaflets that may result in clots.

More specifically, when pumped blood is advanced through vein 600 during normal circulation, blood enters valve 116 through inlet 124 and subjects the interior surface of the inflow side of valve body portion 117 to an inlet fluid pressure PI. With venous applications including valves in the lower extremities, PI ranges from 200 mm Hg to 5 mm Hg. When in the closed configuration having the constricted midsection 121, pressure PI acts only on the inflow side of valve 116 from inlet 124 to apex 120a. When inlet pressure PI equals or exceeds actuation pressure PA, midsection 121 of valve body 117 radially expands to at least partially open valve seat 129 and allow flow there through as shown in FIG. 8. Stated another way, the inlet pressure PI radially expands apexes 120a, 120b to open valve seat 129, such that the conical portions 118a, 118b of valve body 117 assume frustoconical shapes. Under certain inlet pressures, valve 116 may approach a tubular or cylindrical shape in the open configuration. However, midsection 121 need radially expand only to a point sufficient to allow flow through valve seat 129 and thus valve 116 may have a shape resembling an hourglass in the open configuration. Generally, venous valve 116 will expand to permit the flow of blood at a rate of about 0.25 L/min to about 5 L/min when in the open configuration.

Accordingly, when an actuation pressure PA is reached the venous blood is pumped through the at least partially open valve seat 129 of lumen 128 and exits valve 116 through outlet 126. During natural pauses of blood flow, inlet pressure PI ceases and thus the fluid pressure acting on the interior surface of the inflow side of the valve body decreases. When inlet pressure PI is less than actuation pressure PA, valve 116 returns to its closed configuration of FIG. 7 in which midsection 121 is constricted and valve seat 129 closes to prevent venous blood from backflowing through valve 116. When in the closed configuration having the constricted midsection 121, an outlet pressure PO acts on the interior surface of the outflow side of valve 116 from second apex 120b to outlet 126. The fluid outlet pressure PO generally results from gravity which causes blood to backflow into the outflow side of valve 116 through outlet 126. With venous applications including valves in the lower extremities, PO typically ranges from 200 mm Hg to 5 mm Hg. In one embodiment, valve 116 will remain in the closed configuration when subjected to backflow pressures of less than about 10 mmHg.

Valve 116 is constructed from a durable biocompatible material such as silicone that is designed to provide enough resistance to remain in the closed configuration and prevent antegrade blood flow there through, yet flexible enough to allow the pumped blood to transform the valve to the open configuration and allow pumped venous blood to flow there through. Other suitable materials include polymeric materials such as polyurethanes, PEBAX, ePTFE, etc.

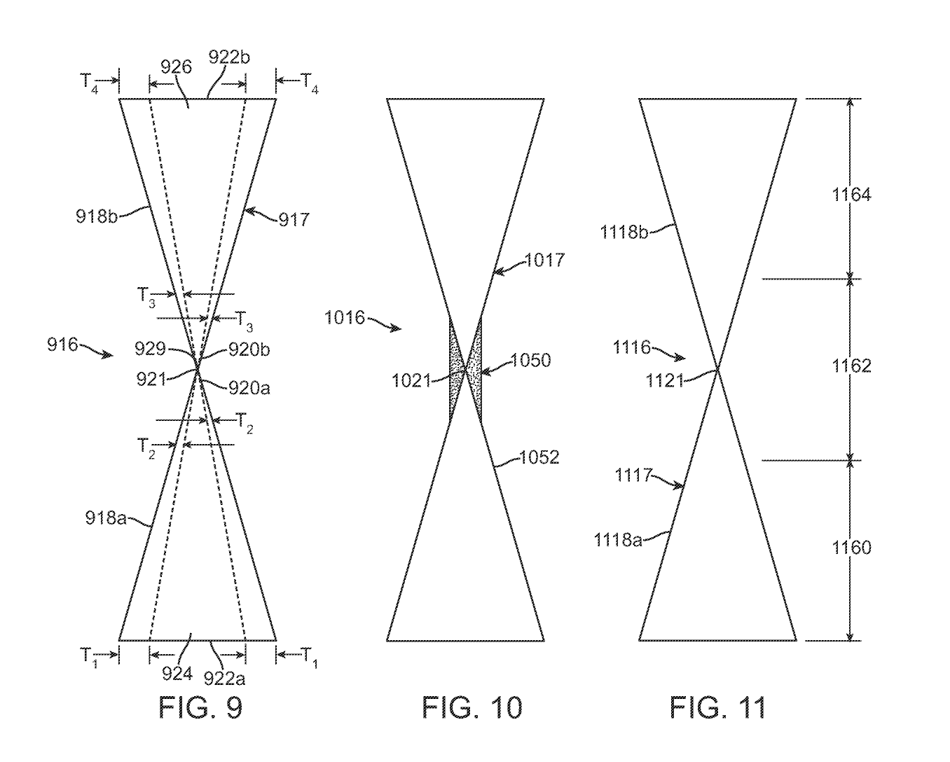

There are several ways to construct the valve prosthesis such that the midsection of the venous valve body portion expands to the open configuration in response to actuation pressure PA. For example, FIG. 9 illustrates one embodiment hereof in which the wall thickness of valve 916 is optimally varied such that midsection 921 will expand to the open configuration in response to actuation pressure PA. First conical section 918a has a tapered wall thickness that continually decreases from a first wall thickness T₁ at first circular base 922a to a second wall thickness T₂ at apex 920a such that the wall thickness becomes thinner as midsection 921 approaches. Similarly, second conical section 918b has a tapered wall thickness that continually increases from a third wall thickness T₃ at apex 920b to a fourth wall thickness T₄ at second circular base 922b such that the wall thickness becomes thicker as outlet 926 approaches. Tapering both first conical section 918a and second conical section 918b as shown in FIG. 9 results in the wall thickness surrounding midsection 921 being relatively thinner, and accordingly less stiff, than the remaining valve body. Stiffness refers to the resistance of an elastic body to deflection or deformation by an applied force. Due to the wall thickness variation, the ends of valve body 917 have a greater stiffness (or more resistance to bending) than relatively thinner midsection 921. Thus, when inlet pressure PI equals or exceeds actuation pressure PA, the relatively thinner and less stiff midsection 921 of valve body 917 will radially expand to at least partially open valve seat 929 and allow flow there through. In one embodiment, wall thicknesses T₁, T₂, T₃, and T₄ may each range between 0.001 inch to 0.012 inch.

In one embodiment, shown in FIG. 9, it may be desirable to form second conical section 918b with a more gradual taper such that the wall thickness of second conical section 918b, when considered as a whole, is generally thinner than first conical section 918a. Particularly, first wall thickness T₁ at first circular base 922a is greater than fourth wall thickness T₄ at second circular base 922b and second wall thickness T₂ at apex 920a is greater than third wall thickness T₃ at apex 920b. Such a construction allows the wall of outlet 926 to be relatively thinner than the wall of inlet 924 to ensure than second conical section 918b will expand in response to the actuation pressure PA and valve seat 929 of valve 916 will be sufficiently open to allow flow there through.

FIG. 10 illustrates another embodiment for constructing the valve prosthesis such that the midsection assumes the open configuration in response to an actuation pressure. Valve 1016 includes an expandable annular band 1050 attached to an outside surface 1052 of valve body portion 1017. In its relaxed or formed configuration, annular band 1050 surrounds valve 1016 to constrain or close the valve seat (not shown) at midsection 1021. However, annular band 1050 is formed from an expandable material that will assume the open configuration in response to an actuation pressure. For example, the expandable annular band 1050 may be formed from nickel-titanium (nitinol) or another superelastic material. Annular band 1050 may have any suitable configuration such as annular bands or sinusoidal patterned wire rings.

FIG. 11 illustrates yet another embodiment for constructing the valve prosthesis such that the valve seat assumes or expands to the open configuration in response to an actuation pressure. Valve 1116 includes first conical section 1118a and second conical section 1118b, similar to the embodiments described above. However, portions of valve 1116 may be constructed to have different stiffness values such that the midsection 1121 is relatively more flexible than the remaining valve body. As previously mentioned, stiffness refers to the resistance of an elastic body to deflection or deformation by an applied force. More particularly, a first end portion 1160 of valve 1116 and a second end portion 1164 of valve 116 are formed with a first stiffness. An intermediate portion 1162 of valve 1116 extends between first end portion 1160 and second end portion 1164, and includes midsection 1121 of valve body portion 1117. Intermediate portion 1162 is formed with a second stiffness that is different than the first stiffness. The first stiffness is greater than the second stiffness to result in a more flexible area surrounding midsection 1121 than the remaining valve body such that the midsection 1121 opens to the open configuration in response to an actuation pressure, such as described above with respect to the embodiment of FIG. 9.

In one embodiment, a first end portion 1160 and a second end portion 1164 are formed with a first material having the first stiffness while intermediate portion 1162 is formed with a second, different material having the second stiffness. End portions 1160, 1164 and intermediate portion 1162 are sealingly coupled and/or joined in order to form the continuous valve body of valve 1116. Any suitable coupling mechanisms or methods may be employed for connecting end portions 1160, 1664 to intermediate portion 1162. For example, the ends of intermediate section 1162 may be bonded to first and second end portions 1160, 1162. Any one of numerous types of bonding may be employed, such as, for example, ultra-violet cure, instant cure, epoxy type, or cyanoacrylate type. Suitable materials for the first, stiffer material include PEBAX or Polyurethane, and suitable materials for the second, more flexible material include silicone or ePTFE.

In another embodiment, cross-linking of the material may be employed in order to alter the modulus of elasticity at end portions 1160, 1164. More particularly, intermediate portion 1162 and end portions 1160, 1164 are integrally formed and/or machined from the same material having the first stiffness. End portions 1160, 1164 are heat treated or irradiated in order to change the modulus thereof and obtain the second stiffness. Suitable materials for this integral, seamless embodiment include, but are not limited to, thermoplastics such as polyethylene or PEBAX.

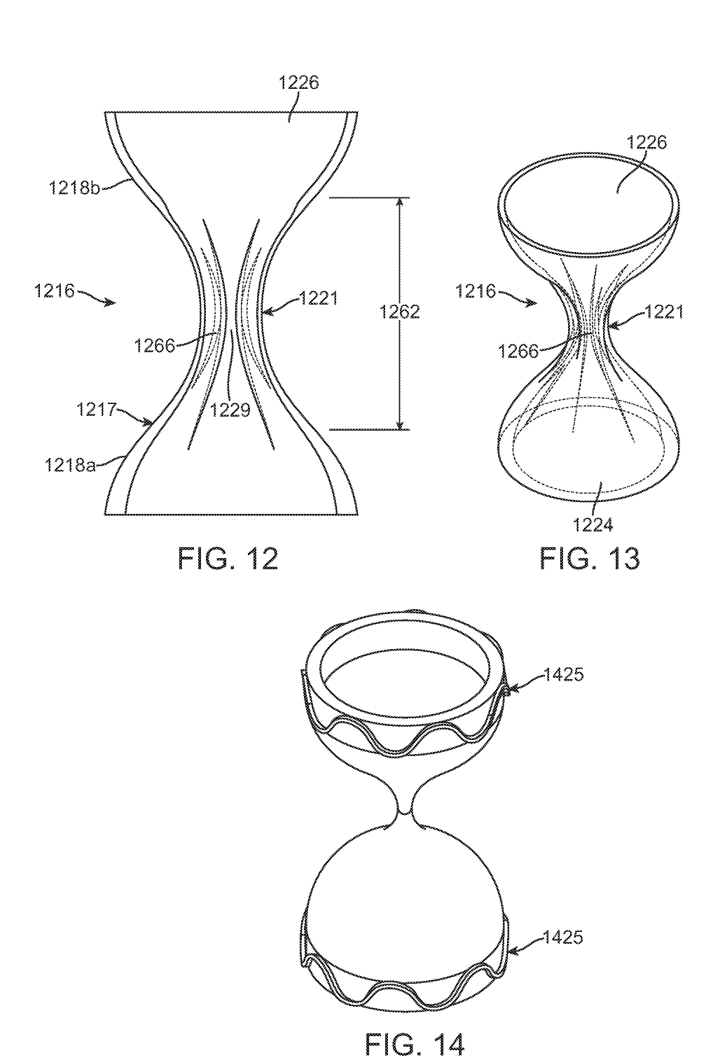

FIGS. 12 and 13 illustrate yet another embodiment for constructing the valve prosthesis such that the midsection assumes the open configuration in response to an actuation pressure. FIG. 12 is a side view of valve 1216 in a closed valve configuration, and FIG. 13 is a perspective view of valve 1216 is a closed valve configuration. Valve 1216 has a valve body 1217 including first conical section 1218a and second conical section 1218b, similar to the embodiments described above. However, valve 1216 includes folds 1266 of the valve body material that open or unfold in response to an actuation pressure. When valve 1216 is expanded, folds 1266 allow valve body 1217 to approach a generally tubular or cylindrical shape to accommodate a large volume of flow through valve seat 1229.

In this embodiment, valve 1216 is integrally formed and/or machined from the same material and folds 1266 are formed within the material at an intermediate portion 1262 positioned between inlet 1224 and outlet 1226 of the valve. In a closed configuration (shown), folds 1266 form a constricted midsection 1221 that prevents flow there through. The intermediate portion 1262 of valve 1216 having folds 1266 has a wall thickness less than the wall thickness of the remainder of the valve body, as may be achieved e.g., by making two different extrusions of the same material or by necking/thinning the valve body at intermediate portion 1262. As such, similar to above embodiments, intermediate portion 1262 is relatively more flexible and less stiff than the remainder of the valve body such that valve seat 1229 may assume or expand to the open configuration in response to an actuation pressure. When pumped blood is advanced during normal circulation, blood enters valve 1216 through inlet 1224 and subjects the interior surface of the inflow side of valve 1216 to inlet fluid pressure PI. When inlet pressure PI equals or exceeds actuation pressure PA, folds 1266 open such that midsection 1221 radially expands to allow flow there through.

The valve prostheses described herein are preferably delivered in a percutaneous, minimally invasive manner and may be delivered by any suitable delivery system. In general, a venous valve prosthesis having one or more self-expanding anchors is loaded into a sheathed delivery system, compressing the self-expanding anchors. As previously described, the self-expanding anchors may have an annular bands configuration as shown in FIGS. 7-8 or may have a sinusoidal patterned configuration as shown in FIG. 14. Optionally, the valve prosthesis may include one or more radiopaque or echogenic markers thereon in order to aid in positioning the valve prosthesis to span across the incompetent native valve. The delivery system is percutaneously introduced into the patient's vasculature. Access to the vasculature may be achieved through a branch of the femoral vein, or alternatively, may be achieved through a branch of the subclavian vein. The delivery system is then threaded or tracked through the vascular system of the patient until venous valve 116 is located within a predetermined target site, an incompetent native valve within a vein. Once properly positioned, the sheath of the delivery system is removed to allow the anchors to self-expand, appose the venous wall, and secure the valve prosthesis inside of the native valve within the vein, thus deactivating the incompetent native valve and surrounding area. Once the venous valve prosthesis is properly positioned at the target site, the delivery system may be retracted and removed from the patient.

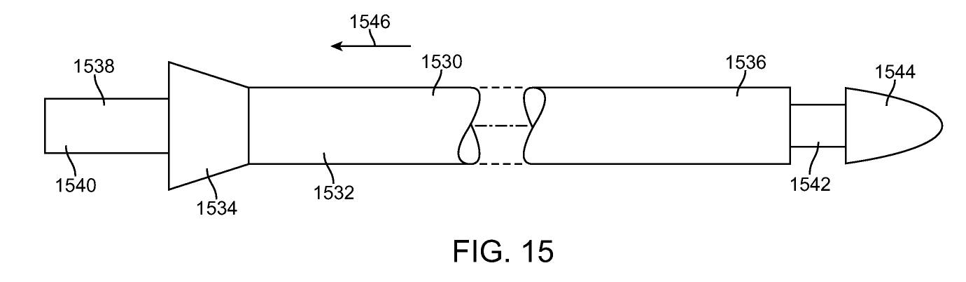

For example, FIG. 15 illustrates a schematic side view of an exemplary delivery system for delivering and deploying a valve prosthesis having one or more self-expanding anchors attached thereto as described above. Self-expanding anchors 125, 1425 effectively make the valve prosthesis a self-expanding conduit. The delivery system includes a retractable outer shaft 1530 having a proximal end 1532 and a distal end 1536, and an inner shaft 1538 having a proximal end 1540 and a distal end 1542. Outer shaft 1530 defines a lumen extending there through (not shown), and inner shaft 1538 slidably extends through the lumen of outer shaft 1530 to a distal tip 1544 of the delivery system. Distal tip 1544 is coupled to distal end 1542 of inner shaft 1538, and may be tapered and flexible to provide trackability in tight and tortuous vessels. In an embodiment, inner shaft 1538 may define a guidewire lumen (not shown) for receiving a guidewire there through or may instead be a solid rod without a lumen extending there through.

The valve prosthesis (not shown in FIG. 15) is mounted on distal end 1542 of inner shaft 1538. The valve prosthesis may be mounted on distal end 1542 of inner shaft 1538 by any suitable manner known in the art, such as self-expanding attachment bands, a cap coupled to the distal end of the inner shaft to retain the valve prosthesis in a radially compressed configuration, and/or the inclusion of slots, ridges, pockets, or other prosthesis retaining features (not shown) formed into the exterior surface of the inner shaft to secure the valve prosthesis in frictional engagement with the delivery system. Outer shaft 1530 covers and constrains the valve prosthesis while the delivery system is tracked through a body lumen to the deployment site. Outer shaft 1530 is movable in an axial direction along and relative to inner shaft 1538 and extends to a proximal portion of the delivery system where it may be controlled via an actuator, such as a handle 1534, to selectively expand the valve prosthesis. When the actuator is operated, outer shaft 1530 is retracted over inner shaft 1538 in a proximal direction as indicated by directional arrow 1546. When outer shaft 1530 is proximally retracted with respect to the hub of the delivery system, the self-expanding valve prosthesis is released and allowed to assume its expanded configuration. An exemplary suitable delivery system is described in U.S. Pat. No. 7,264,632 to Wright et al., which is hereby incorporated by reference in its entirety.

Although the valve prosthesis is described herein as self-expanding for percutaneous placement, it should be understood that the valve prosthesis may alternatively be surgically implanted within a vein in a non-percutaneous manner and may be anchored to the vein in any suitable manner, such as via sutures, clips, or other attachment mechanisms.

While various embodiments hereof have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A one-way venous valve prosthesis for percutaneous placement within a vein comprising:
a valve body having an inlet and an outlet with a lumen that extends there between, the valve body being operable to alternate between
a closed configuration wherein the valve body has a double cone shape with apexes located at a midsection of the valve body, wherein a valve seat is defined within the lumen of the valve body at the midsection thereof, and
an open configuration wherein the valve body assumes a double frustoconical shape,
wherein the valve seat is constricted to prevent blood flow there through when the valve body is in the closed configuration and the valve seat is expanded to allow blood flow there through when the valve body is in the open configuration.

2. The venous valve prosthesis of claim 1, wherein the valve seat expands to the open configuration in response to an actuation pressure and wherein the valve seat closes to the closed configuration in the absence of the actuation pressure.

3. The venous valve prosthesis of claim 1, further comprising:
an annular self-expanding first anchor attached to the inlet; and
an annular, self-expanding second anchor attached to the outlet.

4. The venous valve prosthesis of claim 3, wherein the first and second anchors are nickel-titanium scaffolds.

5. The venous valve prosthesis of claim 1, wherein the valve body is formed from silicone.

6. The venous valve prosthesis of claim 1, wherein the valve body includes a first conical section and a second conical section, and the first conical section has a tapered wall thickness that continually decreases from the inlet to the midsection of the valve body and the second conical section has a tapered wall thickness that continually increases from the midsection of the valve body to the outlet.

7. The venous valve prosthesis of claim 6, wherein the second conical section includes a more gradual taper than the first conical section such that, when considered as a whole, the wall thickness of the outlet is relatively less than the wall thickness of the inlet.

8. The venous valve prosthesis of claim 1, wherein a portion of the valve body adjacent the inlet and a portion of the valve body adjacent the outlet have a first stiffness and an intermediate portion between the inlet and outlet has a second stiffness, wherein the first stiffness is greater than the second stiffness.

9. The venous valve prosthesis of claim 1, further comprising:
an expandable annular band attached to an outside surface of the valve body around the midsection.

10. The venous valve prosthesis of claim 9, wherein the expandable annular band is formed from nickel-titanium.

11. The venous valve prosthesis of claim 1, wherein an intermediate portion between the inlet and outlet includes folds of material of the valve body.

12. A one-way venous valve prosthesis for percutaneous placement within a vein comprising:
a valve body having a first conical section with an inlet and a second conical section with an outlet and a lumen that extends between the inlet and the outlet, the valve body operable to alternate between a closed configuration wherein the valve body has a double cone shape with apexes located at a midsection of the valve body, wherein a valve seat is defined within the lumen of the valve body at the midsection thereof, and an open configuration wherein the valve body has a double frustoconical shape,
wherein the valve seat is constricted to prevent flow there through when the valve body is in the closed configuration and the valve seat is expanded to allow flow there through when the valve body is in the open configuration, and
wherein the first conical section has a tapered wall thickness that continually decreases from the inlet to the midsection of the valve body and the second conical section has a tapered wall thickness that continually increases from the midsection of the valve body to the outlet such that the valve seat expands to the open configuration in response to an actuation pressure and the valve seat closes in the absence of the actuation pressure.

13. The venous valve prosthesis of claim 12, wherein the second conical section includes a more gradual taper than the first conical section such that, when considered as a whole, the wall thickness of the outlet is relatively less than the wall thickness of the inlet.

14. The venous valve prosthesis of claim 12, further comprising:
an annular self-expanding first anchor attached to the inlet; and
an annular, self-expanding second anchor attached to the outlet.

15. The venous valve prosthesis of claim 12, wherein the valve body is formed from silicone.

16. A percutaneous method of repairing an insufficient native valve within a vein, the method comprising the steps of:
percutaneously introducing a delivery system having a valve prosthesis loaded thereon into the patient, wherein the valve prosthesis has a valve body including an inlet and an outlet with a lumen that extends there between, the valve body operable to alternate between a closed configuration wherein the valve body has a double cone shape with a valve seat defined within the lumen of the valve body at a midsection thereof, and an open configuration wherein the valve body has a double frustoconical shape, wherein the valve seat is constricted to prevent flow there through when the valve body is in the closed configuration and the valve seat is open to allow flow there through when the valve body is in the open configuration;
tracking the valve prosthesis to the insufficient native valve; and
implanting the valve prosthesis such that the valve body spans across the insufficient native valve.

17. The method of claim 16, wherein the valve seat expands to the open configuration in response to an actuation pressure and wherein the valve seat closes to the closed configuration in the absence of the actuation pressure.

18. The method of claim 16, wherein the valve prosthesis includes an annular self-expanding first anchor is attached to the inlet and an annular, self-expanding second anchor is attached to the outlet.

19. The method of claim 18, wherein the delivery system includes a retractable sheath and the step of implanting the valve prosthesis further includes retracting the sheath of the delivery system to expand the valve prosthesis within the vein.

20. The method of claim 18, wherein the step of implanting the valve prosthesis includes positioning the valve body to bypass the sinus of the insufficient native valve in order to prevent blood stasis and further deterioration of the insufficient native valve.
